(19) United States
(12) Patent Application Publication
(10) Pub. No.: US 2009/0069762 A1
(43) Pub. Date: Mar. 12, 2009

(54) Guard for bone collection

(76) Inventor: Hesham Alkhateeb, London (GB)

Correspondence Address: STETINA BRUNDA GARRED & BRUCKER, 75 ENTERPRISE, SUITE 250, ALISO VIEJO, CA, 92656, US

(21) Appl. No.: 11/900,560

(22) Filed: Sep. 12, 2007

(51) Int. Cl. A61M 1/00

(52) U.S. Cl. 604/327

(57) ABSTRACT

The guard is arranged to receive the end of a bone which is due to be reshaped or undergo any other procedure that produces fragments of the bone. The guard is provided with at least one wall to prevent sideways moving fragments from exiting the guard and a base for collecting bone fragments. The base is further provided with means through which the bone can be inserted into the guard.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

This invention relates to a guard configured to collect bone fragments produced during a bone resurfacing operation.

Hip resurfacing operations are an alternative to traditional hip replacement surgery and can be used to replace hip joints whilst preserving bone and maintain normal hip biomechanics.

During a hip resurfacing procedure the femoral head is reshaped by removing the arthritic bone from the femoral head. This is achieved using a femoral reamer allowing the appropriate metallic femoral component to be applied onto the femoral head providing a new surface.

As bone is removed from the surface of the femoral head it can be dispersed throughout the surrounding soft tissues during the procedure. This is not desirable as this bone debris can lead to the formation of new bone (Heterotopic Ossification) with detrimental functional outcomes as a result. The new bone formed may cause restriction of movement of the hip or even fuse the hip if they form a link between the pelvis and the femur.

One known method for reducing the amount of bone debris that ends up in the soft tissues is the placement of a swab having an appropriately sized hole in it over the femoral head. The swab acts to catch any bone falling onto it during the reaming process.

However, the swab material is such that it is possible for the threads of the swab to become caught in the reamer causing the swab to be torn and the threads of the swab entangled onto the reamer, decreasing the swabs bone fragment retention, and compromising the procedure.

### BRIEF SUMMARY

According to the invention there is provided a guard configured to collect bone fragments removed from a bone comprising a base configured to receive the bone and at least one wall connected to the base, the wall being situated such that, in use, the wall continuously surrounds the circumference of the bone.

Preferably the base includes sealing means to seal the base around the received bone to prevent bone fragments from falling through the base.

Optionally, the base may include a hole to receive the bone. If the base includes a hole then the hole preferably includes sealing means arranged to provide a seal between an edge of the hole and the received bone to prevent bone fragments from falling through the hole. Preferably the sealing means is a resilient seal to aid insertion of the bone through the hole.

Alternatively the base may include a plurality of projections. The projections may be from the base into a hole formed in the base or extend from the wall of the guard.

Preferably, the guard further comprises means to remove bone fragments from the guard to further minimise bone fragment retention by the tissue. The means to remove bone fragments may be a suction means and may further include a fluid inlet through which fluid is introduced to the guard.

The suction means may also include a collection means to collect the removed bone fragments removed using the suction means.

Optionally, the guard may include an integral pressure release means or a guide means to direct a needle towards the bone.

### BRIEF DESCRIPTION OF THE DRAWINGS

Other aspects and features of the present invention will become apparent to those ordinarily skilled in the art upon review of the following description of specific embodiments of the invention in conjunction with the accompanying figures.

FIG. 1 illustrates a guard in accordance with a first embodiment of the present invention;

FIG. 2 illustrates a guard in accordance with a first embodiment of the present invention into which a femur has been inserted; and

FIG. 3 illustrates a guard in accordance with a second embodiment of the present invention.

### DETAILED DESCRIPTION

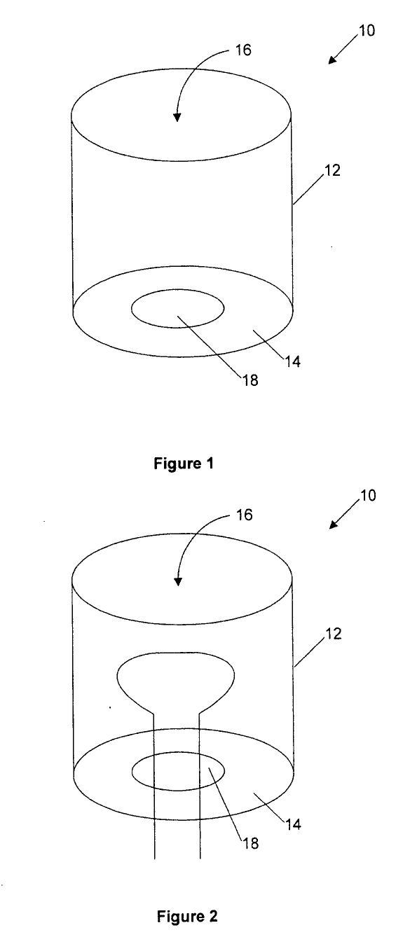

FIG. 1 illustrates one embodiment of the guard 10 of the present invention. The guard 10 has a wall 12 and a base 14. The wall surrounds a space 16 and is sized such that the space 16 can readily accept a head of a femur which is to be resurfaced.

The base 14 of the guard 10 includes a hole 18. Prior to the resurfacing procedure the guard 10 is positioned over the femoral head and lowered with the femoral head and neck proceeding through the hole 18 in the base 14 as illustrated in FIG. 2. In this way the femoral head is positioned within the space 16.

Once the femoral head is positioned within the guard 10 the resurfacing operation can begin. During the resurfacing operation bone fragments are produced as described previously. The bone fragments are prevented from exiting the guard 10 by the guard wall 12 and are collected on the base 14 of the guard 10.

It is advantageous for the base of the guard 10 to be flexible so that it can readily accommodate the head of the femur being inserted through the hole in the base without cracking and thereby reducing the collecting properties of the guard.

The hole in the guard's base may be sized according to the size of the neck or head of the femur to be inserted through it.

Optionally, the guard may be provided with a resilient seal, such as an O-ring, around the edge of the hole in the base. The seal acts to prevent any bone fragments from escaping the guard through any gap between the edge of the hole and the femur neck. The thickness of the seal may be altered according to the size of the femur neck to be accommodated relative to the size of the hole. Preferably, the seal is made from an elastomer such as a rubber but any resilient material may be used.

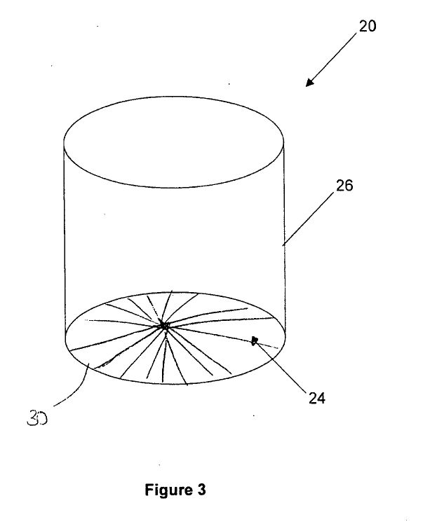

In a second embodiment of the present invention there is provided a guard 20 having a wall 22 and a base 24 as illustrated in FIG. 3. As discussed above the wall 26 of the guard 20 encloses a space 28 into which the head of the femur can be inserted. The base 24 comprises a plurality of overlapping projections 30. The projections 30 are connected perpendicular to, and around, the inner circumference of the wall with their free ends being situated at the centre of the base of the guard.

The projections 30 preferably overlap and are arranged such that they flex to allow a femur head to be passed through the base of the container. Once the femur head has been inserted through the base, the projections are preferably configured to remain in contact with the neck of the femur thereby forming a seal with the femur neck. The seal acts to reduce the loss of bone fragments through the base of the guard.

The projections 30 may be any suitable shape for providing the function of collecting bone fragments and receiving a femur head through them. They may extend to cover the entirety of the base of the guard. Alternatively, the projections may extend only partially across the radius of the guard, thereby leaving a hole in the centre of the projections. The femoral head can then be inserted through the hole. In a further alternative the base may be provided with a hole from which the projections extend to cover the hole and thereby forming a seal against a bone inserted into the hole

The projections may, optionally, extend from the guard wall at a different angle to that described above to aid, for example, insertion of the femoral head or the seal between the femoral neck and the projections.

Preferably, the guards of either embodiment are made from a plastic such as polypropylene or polyvinyl chloride. However, as will by understood by the skilled man, any other suitable material may be used.

Further adaptations may be made to both of the above embodiments to improve bone fragment collection using them.

Firstly the wall of the guard may be provided with a hole or hollow projection to which a pipe can be attached. The pipe is attached to suction means which exerts a force on the bone fragments towards the hole or projection, thereby removing the bone fragments from the guard. The removed bone fragments can then be collected remotely from the guard using any suitable means and disposed of separately to the guard.

The wall of the guard may, in addition to the suction means, be provided with a fluid inlet. Water or any other suitable fluid can then be introduced into the guard through the inlet. The bone fragments collected by the guard will then become suspended in the fluid and can be removed with the fluid using the suction means. The presence of fluid facilitates removal of the bone fragments using the suction means.

Optionally, the guard may be provided with guide means for a venting device. In hip resurfacing when the metal head is cemented to the reshaped femoral head there is increased intramedullary pressure which can cause embolization of fat, bone marrow, cement, and air into the circulation which is known to activate clotting and microthrombus formation in the lungs, and cardiovascular instability with potential lethal consequences.

In order to ameliorate this it is standard practice to insert a venting device into the lesser trochanter to allow pressure to be released. In order to aid the insertion of the venting device into the femur the guard may be provided with guide means allowing accurate insertion of the venting device.

Commonly, the venting device is a metal trocar attached to suction, however any suitable device, such as a syringe, hypodermic needle or canulae may be used.

Alternatively, the guard may be manufactured with an integral venting device to aid this process further. The guide means or integral venting device may be present on a skirt which extends below the base of the container in order to ensure correct placement.

As will be understood by one skilled in the art, the guard may receive any bone end that requires reshaping or from which bone fragments may be produced.

Additionally, the guard does not have to have a cylindrical shape but may have any shape or number of walls desired.

Optionally, the guard may not be formed as a single piece but may, for example, come as two or more semi-cylindrical, or other shaped, portions which are placed around the femoral head and then sealed together to form a guard around the femoral head. The guard pieces may be sealed together through attachments on either end that lock the two pieces together forming a tight seal.

What is claimed is:

1. A guard configured to collect bone fragments removed from a bone comprising:
i) a base configured to receive the bone; and
ii) at least one wall connected to the base;
the wall being situated such that, in use, the wall continuously surrounds the circumference of the bone.

2. A guard as claimed in claim 1 wherein the base includes sealing means to seal the base around the received bone.

3. A guard as claimed in claim 1 wherein the base includes a hole to receive the bone.

4. A guard as claimed in claim 3 wherein the hole has sealing means arranged to provide a seal between an edge of the hole and the received bone.

5. A guard as claimed in claim 4 wherein the sealing means is a resilient seal.

6. A guard as claimed in claim 5 wherein the resilient seal is made from rubber.

7. A guard as claimed in claim 1 wherein the base includes a plurality of projections.

8. A guard as claimed in claim 7 wherein the plurality of projections extend from the wall of the guard.

9. A guard as claimed in claim 7 wherein the base includes a hole to receive the bone and the plurality of projections extend from the edge of the base forming the hole.

10. A guard as claimed in claim 1 wherein the guard further comprises means to remove bone fragments from the guard.

11. A guard as claimed in claim 10 wherein the means to remove bone fragments includes suction means.

12. A guard as claimed in claim 11 wherein the means to remove bone fragments further includes a fluid inlet.

13. A guard as claimed in claim 10 wherein the suction means includes a collection means to collect the removed bone fragments removed using the suction means

14. A guard as claimed in claim 1 further including integral pressure release means.

15. A guard as claimed in claim 1 further including a guide means to direct a needle towards the bone.

16. A guard as claimed in claim 1 wherein the guard is made from plastic.

17. A guard as claimed in claim 1 wherein the bone is a femur.

18. A guard as claimed in claim 1 wherein the bone fragments are collected during hip resurfacing.
